(19) United States
(12) Patent Application Publication
(10) Pub. No.: US 2024/0125685 A1
(43) Pub. Date: Apr. 18, 2024

(54) METHOD AND DEVICE FOR ANALYZING A LIQUID LIABLE TO CONTAIN AN ANALYTE

(71) Applicant: Magia Diagnostics, Echirolles (FR)

(72) Inventors: Paul Kauffmann, Echirolles (FR); Damien Kirk, Echirolles (FR); Mario Fratzl, Echirolles (FR); Roman Gorbenkov, Echirolles (FR)

(21) Appl. No.: 18/546,812

(22) PCT Filed: Feb. 21, 2022
(86) PCT No.: PCT/FR2022/050308

§ 371 Date: Aug. 17, 2023

(30) Foreign Application Priority Data
Feb. 24, 2021 (FR) 2101771

(51) Int. Cl. G01N 15/06; G01N 33/543; G01N 33/58

(52) U.S. Cl. CPC G01N 15/0612; G01N 33/54326; G01N 33/54346; G01N 33/582

(57) ABSTRACT

A method for analyzing a liquid liable to contain an analyte comprises acquiring a digital image of an area to be analyzed using an image-capturing device having an optical axis directed toward the area to be analyzed, the digital image being exposed for an exposure time and exhibiting a spatial variation in intensity that corresponds to a detection pattern when the analyte is present in the sample. The digital image is processed to identify the spatial variation in intensity therein. The medium to be analyzed is placed in a magnetic field referred to as the “illumination” magnetic field during at least some of the exposure time, the illumination magnetic field being produced by an illumination magnetic source. The illumination magnetic field lies parallel to the optical axis of the image-capturing device over at least some of the area to be analyzed. A device is configured for performing such a method.

### CROSS-REFERENCE TO RELATED APPLICATIONS

This application is a national phase entry under 35 U.S.C. § 371 of International Patent Application PCT/FR2022/050308, filed Feb. 21, 2022, designating the United States of America and published as International Patent Publication WO 2022/180334 A1 on Sep. 1, 2022, which claims the benefit under Article 8 of the Patent Cooperation Treaty of French Patent Application Serial No. FR2101771, filed Feb. 24, 2021.

### TECHNICAL FIELD

The technical field of the present disclosure is that of biological analysis in order to detect the presence and/or concentration of an analyte in a sample of liquid, particularly of a biological liquid. The present disclosure relates more particularly to a method for detecting the presence and/or concentration (and more succinctly “analysis”) of an analyte in a sample of biological fluid. This method may be implemented in a portable analysis device of the “Point of Care” type, that is to say making it possible to carry out and interpret a test on-site to make an immediate clinical decision, at the patient's bedside rather than in a central laboratory. The device performs the analysis on a sample collected on an analysis support, such as a microfluidic cartridge.

### BACKGROUND

Document EP3447492 discloses a method for capturing and detecting a species, often referred to as an “analyte,” in a sample of a liquid, particularly of a biological liquid. The principles for capturing and detecting patterns implemented by this method are also explained in the article by Fratzl et al, “Magnetophoretic induced convective capture of highly diffusive superparamagnetic nanoparticles,” Soft Matter, 14. 10.1039/C7SM02324C. They are also presented in the document “Rapid immunoassay exploiting nanoparticles and micromagnets: proof-of-concept using ovalbumin model,” by Delshadi S et al, Bioanalysis. 2017 March; 9(6):517-526. According to this method, the sample is mixed with magnetic particles of nanometric or, more generally, submicrometer size, respectively coupled to capture elements capable of binding to the species whose presence is to be detected or quantified. The species to be detected, the analyte, may be an antigen and the element an antibody, but the reverse configuration is also possible.

Detection elements are also introduced into the sample, for example, a detection antibody or antigen carrying a photoluminescent marker, for example, fluorescent.

By the end of this step, in the solution, complexes formed of the capture element, the analyte and the detection element are thus formed, which are then immobilized on a support comprising magnetic micro-sources ordered according to a specific spatial pattern. The pattern is defined by strong magnetic field zones and weak magnetic field zones inducing significant magnetic field gradients. The complexes entrained by the magnetic particles tend to agglomerate on the support at the zones where the norm of the magnetic field is maximum. The photoluminescent (and especially fluorescent) markers can make the specified spatial pattern apparent, which marks the presence of the analyte in the solution. The mean (spatially) intensity of this light pattern is usually referred to as “specific signal.”

In most cases, and particularly when the analyte is absent from the sample or when its quantity is limited in the sample, the unbound detection elements bearing the photoluminescent markers remain dispersed in suspension in the solution. They contribute to forming a relatively homogeneous light background. The mean (spatially) intensity of this light background forms a signal called “signal of the supernatant.” Besides the unbound photoluminescent markers, this light background is also formed by the light intensity emitted by all the photoluminescent materials of the sample. The capture elements not bound to the analyte and to the detection element are also immobilized on the support, but do not carry markers; they do not contribute to the light pattern or to the light background.

The spatial arrangement in the plane of the support of the magnetic field microsources and the light intensity of the patterns exposed by the photoluminescent markers make it possible to carry out a detection and a quantification of the analyte in the sample without washing, that is to say without eliminating the liquid solution after having immobilized the complexes on the surface of the support, which is particularly advantageous. To enable this detection, the sample and the surface of the support are illuminated in order to allow the detection of the photoluminescent markers, and the acquisition of a digital image is carried out. This digital image therefore has a spatially variable intensity (in the plane of the image) depending on the intensity of the magnetic field produced by the support. The image is processed to identify this spatial variation, and to determine the specific signal and the signal of the supernatant, and the specific signal/signal of the supernatant ratio makes it possible to conclude that the analyte is present in the sample or even to estimate the concentration thereof.

The simplicity of this approach, and, in particular, the absence of a washing step, allows its integration into an autonomous, portable or transportable immunological analysis device “at the patient's bedside,” in the field and without a pump or valve, whereas traditionally this type of analysis is conducted in a central laboratory.

It is generally sought to lower the detection limit of the analyte as much as possible in the sample. This leads to the processing of low-contrast images, and the processing must be very sensitive (to avoid false negatives, that is to say concluding that the analyte is not in the sample when it was in fact present in a low concentration) and very specific (to avoid false positives, that is to say detecting the presence of the analyte in the sample when it wasn't there). Generally and for a given analyte concentration in the sample, it is sought to form images having a high contrast, that is to say having a spatial variation in intensity with high amplitude, in order to make the analysis more reliable.

### BRIEF SUMMARY

One aim of the present disclosure is to provide at least a partial solution to this problem. More precisely, an object of the present disclosure is to provide an analyzing method and an analysis device capable of producing a digital image of the surface of the support having, for a given analyte concentration in the sample, an improved contrast relative to images produced according to the prior art.

In order to achieve this aim, the subject matter of the present disclosure proposes a method for analyzing a liquid that may contain an analyte, a sample of the liquid being arranged on an area to be analyzed of an analysis support comprising a rear face opposite the area to be analyzed, the area to be analyzed having a plurality of attraction zones arranged according to a detection pattern. The sample comprises magnetic complexes comprising the analyte and a photoluminescent marker immobilized at the attraction zones, and/or supernatant photoluminescent markers. The analyzing method comprises a step of acquiring a digital image of the area to be analyzed during an exposure time, using an image capturing device having an optical axis directed toward the area to be analyzed, the digital image having a spatial variation in intensity in accordance with the detection pattern when the analyte is present in the sample. The analyzing method also comprises a step of processing the digital image to identify the spatial variation in intensity therein.

The method is remarkable in that, during at least part of the exposure time, the analysis support is arranged in a magnetic field called an “illumination” field produced by an illumination magnetic source, the illumination magnetic field being parallel to the optical axis over at least part of the area to be analyzed.

According to other advantageous and non-limiting features of the present disclosure, either individually or in any technically feasible combination:
the illumination magnetic source is operable to place the analysis support in the illumination magnetic field;
the analyzing method comprises a step of moving the analysis support relative to the illumination magnetic source in order to selectively place the analysis support within and outside the illumination magnetic field;
the movement of the analysis support relative to the illumination magnetic source is controlled so as not to change the direction or reverse the orientation of the field at the area to be analyzed during this movement;
the movement of the analysis support relative to the illumination magnetic source is controlled so that the orientation of the illumination field performs at least one full rotation;
the acquisition step comprises a plurality of exposure periods to establish, respectively, a plurality of digital images, and the method comprises, between two exposure periods, a positioning step during which the relative position of the magnetic source of illumination with respect to the analysis support is modified;
the analyzing method comprises, before the acquisition step, a step of attraction of the magnetic complexes optionally present in the sample to immobilize them at the attraction zones;
the attraction step comprises exposing the analysis support to an attraction magnetic field provided by the illumination magnetic source;
the attraction step comprises exposing the analysis support to an attraction magnetic field produced by an attraction magnetic source, distinct from the illumination magnetic source;
the attraction magnetic field and the illumination magnetic field have the same direction and orientation at the area to be analyzed.
the analysis support comprises a magnetic layer at least partly defining the attraction zones and a non-magnetic surface film arranged on the magnetic layer, the magnetic surface film defining the area to be analyzed.

According to another aspect, the subject matter of the present disclosure proposes an analysis device comprising:
a host support for receiving and placing in the acquisition position an area to be analyzed of an analysis support and a plurality of attraction zones arranged according to a detection pattern on the area to be analyzed;
an image capturing device having an optical axis and a depth of field, the image capturing device being arranged to receive the area to be analyzed in its depth of field when the analysis support is in the acquisition position;
an illumination magnetic source capable of producing a magnetic field called an “illumination” field to which the analysis support is exposed when that support is in the acquisition position, the illumination magnetic field being parallel to the optical axis over at least part of the area to be analyzed.

According to other advantageous non-limiting features of this aspect of the present disclosure, taken alone or according to any technically feasible combination:
the image capturing device is arranged on one side of the host support, and the illumination magnetic source is arranged on the other side of the host support;
the illumination magnetic source is movable to selectively place the host support in the illumination magnetic field or place the host support outside the illumination magnetic field;
the analysis device comprises an attraction magnetic source, distinct from the illumination magnetic source, to produce an attraction magnetic field;
the analysis device comprises a transfer rail for moving the analysis support from an incubation position where it can be subjected to the field produced by the attraction magnetic source to the acquisition position where it can be subjected to the illumination field produced by the illumination magnetic source;
the device further comprises an analysis support arranged on the host support, the analysis support comprises a magnetic layer at least partly defining the attraction zones and a non-magnetic surface film arranged on the magnetic layer, the magnetic surface film defining the area to be analyzed.

### BRIEF DESCRIPTION OF THE DRAWINGS

Other features and advantages of the present disclosure will emerge from the following detailed description of embodiments of the present disclosure with reference to the accompanying figures, in which:

FIGS. 1 and 2 show, in perspective and in an exploded view, a cartridge forming a preferred example of an analysis support allowing the implementation of a method according to the present disclosure;

FIG. 3 shows a sectional view, in the analysis chambers, of the cartridge shown in FIGS. 1 and 2;

FIG. 4 schematically shows, in plan view, a detection pattern defined by the magnetization produced by a magnetic layer integrated into the support of a cartridge, the magnetic field present in an analysis chamber and the norm of this field;

FIG. 5 shows the main steps of a method according to the present disclosure;

FIG. 6 shows an image of an area to be analyzed acquired during a method according to the present disclosure;

FIG. 7 shows the application of an illumination magnetic field to a support during a step of acquisition of a method according to the present disclosure;

FIGS. 8A and 8B represent two possible configurations of an illumination magnetic source;

FIG. 9 shows a mobile illumination magnetic source;

FIG. 10 shows an analysis device according to one embodiment;

FIG. 11 shows the benefit provided by the application of an illumination magnetic field according to the present disclosure; and

FIG. 12 shows an illumination magnetic source that can be brought in a direction perpendicular to an area to be analyzed so as to avoid or limit the immobilization of the complexes outside detection patterns.

### DETAILED DESCRIPTION

Analysis Medium

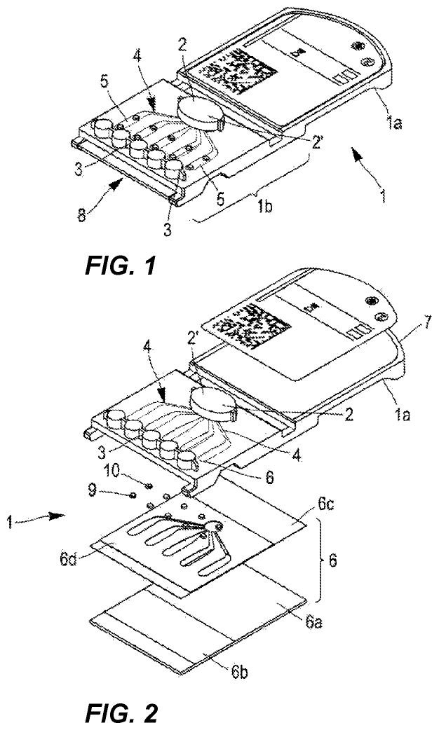

FIGS. 1 and 2 show a cartridge 1 for receiving samples of a liquid, typically a biological liquid, which is likely to contain an analyte that is to be detected or whose concentration is desired to be determined. For the sake of brevity, the term “analysis” refers to the steps of detecting the analyte and/or the steps of determining its concentration in the liquid sample. The cartridge 1 shown in these figures forms a preferred, but in no way limiting example, of implementing an analysis support in a method according to the present disclosure, this analysis support being intended to receive the sample to be analyzed.

This cartridge 1 comprises a gripping end 1a, which makes it possible to manipulate the cartridge 1. The gripping end of the cartridge here bears a label, arranged on the side of the upper face of the cartridge and, in particular, making it possible to identify the cartridge using an identification mark such as, for example, a barcode or a two-dimensional code, allowing identification and traceability of the analyses carried out by means of the analysis cartridge 1 in question. The identification means may alternatively comprise an “RFID” chip.

The cartridge 1 also comprises a microfluidic part 1b. This part extends along a main plane intended to be positioned horizontally. As shown in FIGS. 1 and 2, the microfluidic part 1b comprises a pour opening 2 for pouring the biological liquid into the cartridge 1, for example, via a pipette. The opening 2 opens into an array of channels 4, extending in the main plane of the cartridge 1 and allowing the flow and distribution of the biological liquid into a plurality of analysis chambers 5 via channels, called “upstream” channels of the array of channels 4.

The array of channels 4 of the cartridge 1 also comprises vent channels, which fluidly and respectively connect the analysis chambers 5 to vents 3, these vents making it possible to force the air from the fluid array of the cartridge 1 as the biological liquid progresses into this array.

The sample analyzed is formed of the biological liquid that fills a chamber 5, and the shown cartridge 1 therefore makes it possible to conduct a plurality of analyses on the biological liquid, an analysis being able to be independently conducted on the samples respectively held in the chambers 5. The opening 2, the vents 3 and the array of channels 4, connecting the opening 2 to the vents 3 define a plurality of analysis paths of the cartridge 1. It would naturally be possible to provide a cartridge containing only a single analysis chamber 5, although the ability to have a plurality of analysis chambers in one cartridge is particularly advantageous.

In the example shown in FIGS. 1 and 2, the opening 2 is surmounted by a reservoir 2′, projecting from an upper face of the cartridge 1. The reservoir has sufficient capacity to hold a volume of biological liquid at least equal to the volume of the fluid array of the cartridge 1 (that is to say, the array of channels 4, including the analysis chambers 5 and the vent channels). This volume may typically be between 5 mm 3 and 500 mm³, and, more specifically, between 20 mm 3 and 100 mm³.

Similarly, the vents 3 are respectively surmounted by peripheral walls in order to retain any excess volume of biological liquid, according to the principle of communicating vessels. Advantageously, these walls having a height at least equal to the height of the reservoir 2′ in order to prevent the liquid from escaping from the cartridge, which could pose health problems, or even damage an analysis device wherein the cartridge is intended to be inserted.

By way of illustration, the cartridge 1 can have a size of between 2 cm and 10 cm in width and in length, and have a thickness of between 4 mm and 10 mm. Each chamber 5 may have a volume typically between 1 mm 3 and 50 mm³ in order to receive the sample, advantageously between 5 mm 3 and 25 mm³.

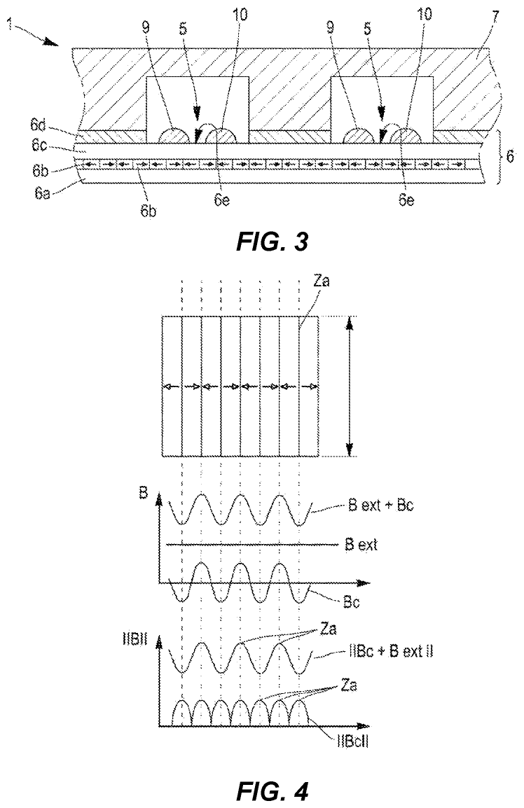

The cartridge 1 is formed of an analysis support 6 and an upper cover 7 covering the support. The support 6 and the upper cover 7 are assembled together by placing their surfaces, referred to as “main” surfaces, facing one another. The fluid array (channels, chamber, etc.) of the cartridge 1 is defined by recesses formed on the main surface of the analysis support 6 and/or on the main surface of the upper cover 7, that is to say on the faces of these two elements that are intended to be assembled together. The main surface of the analysis support 6 therefore constitutes the bottom of the cartridge analysis chambers 5, and each of these bottoms will be referred to as an area to be analyzed 6e (visible in FIG. 3) in the rest of this description. In order to be complete, the analysis support 6 also has a so-called “rear” face 8, opposite its main surface, which carries the area to be analyzed(s) 6e of the cartridge 1.

The upper cover 7, at least for the part that overhangs the analysis chambers 5, is formed of a transparent material in the emission wavelength range of the photoluminescent markers when the cartridge is used for the immunological analysis presented in the background of the present disclosure. It may be a plastic material, for example, based on polycarbonate, cyclo-olefin copolymer or polystyrene. It may also be glass. The outer surface of the upper cover 7 is preferentially optically polished at least in front of the analysis chambers 5. These features enable and promote the optical analysis of the samples of biological liquid contained in the chambers 5, as will be explained in a subsequent section of this description.

The fluid array therefore extends in the main plane of the cartridge. It is of millimetric size, that is to say that the width of the channels 4 of the array and of the analysis chambers 5 is typically between 0.1 mm and 10 mm. The height of these elements, that is to say their extent in a direction perpendicular to the main plane of the cartridge 1, is also millimetric, between 0.1 mm and 10 mm. The biological liquid propagates in this array by capillary action.

Of course, it is possible to provide a cartridge comprising a simpler or more complex fluid array than the one used in the example. Thus, an analysis path of the cartridge may include chambers other than the analysis chamber 5, such as, for example, one or a plurality of incubation chambers arranged upstream of the analysis chamber 5. These incubation chambers may comprise reagents distinct from those with which the fluid mixes before being transported into the analysis chamber 5. The array of channels 4 can therefore also be more complex than the one shown in the figures, and extend into each analysis path, from the opening 2 to the vent 3, by fluidly connecting the different chambers according to any conceivable configuration. In an alternative configuration to that shown in FIG. 1, provision could be made for the cartridge to have a plurality of openings, for example, an opening dedicated to each chamber of the cartridge.

With reference to FIG. 2, the analysis support 6 is composed of a rigid substrate 6a comprising a layer or a magnetic zone 6b. The substrate 6a can be formed of a plastic material. The magnetic layer/zone 6b can be arranged on the substrate 6a, or integrated into this substrate, at least at the analysis chambers 5 of the fluid array. The magnetic layer/zone 6b does not necessarily cover the entire surface of the substrate 6a.

The magnetic layer 6b is typically composed of magnetic composite materials, such as ferrites, randomly distributed in a polymer or oriented along a pre-orientation axis. It may be hard ferromagnetic composite materials, having a coercivity of between 0.01 T and 0.5 T, advantageously between 0.25 T and 0.4 T. This magnetic layer may be similar to a conventional magnetic recording strip.

The substrate 6a comprises a non-magnetic surface film 6c (or a plurality of such films) covering the magnetic layer 6b, and, more generally, the substrate 6a. This non-magnetic surface film, with a thickness that may be between 10 and 100 microns, for example, aims to move the magnetic layer 6b away from the bottom (analysis surfaces 6e) of the analysis chamber 5. The surface of the non-magnetic surface film exposed in the chambers 5 of the cartridge 1 forms the analysis surfaces 6e of those chambers 5. In order not to disturb the measurement, the non-magnetic surface film 6a has a low autofluorescence. For reasons of clarity, “non-magnetic” denotes a material whose magnetic susceptibility is very low, less than 10′, such as a paramagnetic or diamagnetic material. The non-magnetic surface film 6c may, for example, be formed from a plastic material, such as polypropylene.

In addition to the substrate 6a, the analysis support 6 of FIG. 2 also comprises an adhesive interlayer film 6d arranged on the non-magnetic surface film 6c. The interlayer film 6d of FIG. 2 has a cutout according to a pattern corresponding to the array of upstream channels 4 and to the analysis chambers 5 and to the opening 2. In general, the interlayer film 6d has cutouts aimed at defining at least a portion of the fluid array of the cartridge. The interlayer film 6d also allows the upper cover 7 to be assembled and hermetically sealed to the support 6 at their surfaces in contact. It may be a double-sided adhesive film. As is well known per se, such a film consists of a strip, for example, plastic, both faces of which are coated with an adhesive material.

It is naturally possible to envisage other means for defining the fluid network of the cartridge 1 than to provide the analysis support 6 with a pre-cut interlayer film. Whether such a film is present or not, the cartridge 1 can be constituted by assembling the analysis support 6 to the upper cover 7. It is also noted that in general, it is not necessary to provide the support 6 with an upper cover, although this embodiment is preferred.

Referring to the description of the magnetic nature of the cartridge, the magnetic layer 6b comprises a succession of polarized regions having different orientations and/or directions (preferentially of the same direction but of opposite orientation as illustrated in FIG. 3). As shown in FIG. 4, which represents in top view the portion of the magnetic layer 6b forming (with the non-magnetic surface film 6c) the bottom of a chamber 5, i.e., the area to be analyzed 6e, the magnetically polarized regions extend in lines in a main direction in the example shown.

At the interfaces between two different polarization zones, regions of relatively strong magnetic intensity on the area to be analyzed, i.e., the bottom of the analysis chamber 5. These regions form attraction zones of the area to be analyzed. The gradients on the surface of the non-magnetic surface film 6c may have a typical value of between 5 T/m and 1000 T/m, preferentially 50 T/m and 150 T/m. The attraction zones are therefore arranged in the form of a plurality of lines Za running along the main direction. The particular arrangement of these lines defines, in combination, a detection pattern.

It is understood that the arrangement of lines shown in this example illustrates one particular form of a detection pattern. A cartridge 1 is more generally provided with magnetically polarized regions defined in each analysis chamber 5. A well-determined detection pattern is desirable, but the configuration of the pattern can be freely chosen.

FIG. 4 also shows the field Bc generated on the area to be analyzed 6e of a chamber 5 by the magnetic layer 6b and the norm of this field. As will be explained below, it may be useful to add an additional external field Bext to the field produced by the layer 6b. FIG. 4 shows this external field Bext, which combines with the field Bc produced by the layer 6b and the norm of this combined field. It is observed that the application of this external magnetic field Bext can lead to eliminating certain attraction zones Za produced when only the field provided by the magnetic layer 6b is present. However, in every case, these attraction zones are arranged along lines Za parallel to the main direction P, or, more generally, according to a detection pattern, the features of which are fully determined.

In the case of a chamber 5 having the dimensions indicated above, it is possible to consider forming a detection pattern comprising between 2 and 50 lines, the lines having a thickness of between 1 micron and 150 microns (advantageously between 5 microns and 30 microns) and separated from each other by a spacing of between 5 microns and 300 microns, advantageously between 25 microns and 200 microns.

With reference to FIG. 3, and according to a particular embodiment, the cartridge 1 has been advantageously prepared to place in each chamber 5 a controlled quantity of magnetic particles of nanometric dimensions, typically between 25 nm and 500 nm, and preferentially between 100 and 300 nm. In a particular example, these particles have a dimension of 200 nm. These particles are typically in the form of beads having superparamagnetic features and are biocompatible. They may, in particular, be covered with a polymer (of polystyrene type) having a surface treatment that allows them to be functionalized with type Ac or Ag proteins. This functionalization could also correspond to the grafting of DNA or RNA strands. The magnetic particles are bound to capture agents capable of associating with the analyte. This association is referred to as “capture elements.” The controlled quantity of the capture elements 9 is such that their concentration in the volume of the chamber once filled with the biological fluid is between 10⁶ particles/mL and 10¹² particles/mL, and advantageously between 10⁸ particles/mL and 10⁹ particles/mL. The controlled quantity of the capture elements 9 is here arranged in the form of a cluster formed of magnetic nanoparticles held together, and on which capture agents are grafted, the capture agents being configured to specifically bind with the analyte. This cluster is adhered to the area to be analyzed 6e of the chamber 5, that is to say on the non-magnetic surface film 6c forming the bottom of this chamber. The term “magnetic nanoparticles held together” means a set of nanoparticles linked together, the cohesion between these nanoparticles potentially being direct or indirect. Direct cohesion may, in particular, be provided by dry or freeze-dried nanoparticles, while indirect cohesion can be ensured by an encapsulation material. In this respect, the encapsulation material may comprise sugar (trehalose, glucose, etc.) or viscous solution (for example, TWEEN®), or glycerol. The retention of the nanoparticles between them, and in the form of clusters, ensures better stability thereof over time. The implementation of an encapsulation material makes it possible to facilitate the suspension of nanoparticles presented below in the rest of the description.

Similarly, the chambers 5 advantageously each contain a cluster of detection elements 10 adhering to the bottom of these chambers. These detection elements 10 are also capable of binding to the analyte, and they carry photoluminescent markers, for example, fluorescent markers.

The clusters of capture 9 and detection 10 elements are also visible in FIG. 2. They can be made adherent to the support 6 at locations corresponding to the position of the analysis chambers 5, before the upper cover 7 is placed on the support 6. Recesses of the support 6 can be used, which, in particular, define the cavity of the chambers 5, in order to identify these locations.

Provision may be made for each chamber 5 of a cartridge 1 to be prepared to receive capture elements 9 and/or detection elements 10 of different natures, so as to carry out multiple analyses of the biological liquid introduced into the cartridge 1. Provision may also be made for the detection pattern encoded by the portion of the magnetic layer 6b that is arranged at a chamber 5 to be different from one chamber to the other.

Use of the Cartridge for the Capture and Detection of the Analyte

When the biological liquid to be analyzed is introduced into the cartridge 1, the liquid flows into the array of channels 4 to fill the analysis chambers 5 and propagates into the vent channels.

The following capture and detection steps are preferably applied to each chamber 5 individually, successively, when the cartridge 1 has such a plurality of chambers 5 rather than collectively. The duration of each of these steps is thus controlled for each sample contained in a chamber 5, and therefore the analysis is precise. However, it is not totally excluded that these steps, or some of them, may be applied collectively to a plurality of chambers 5.

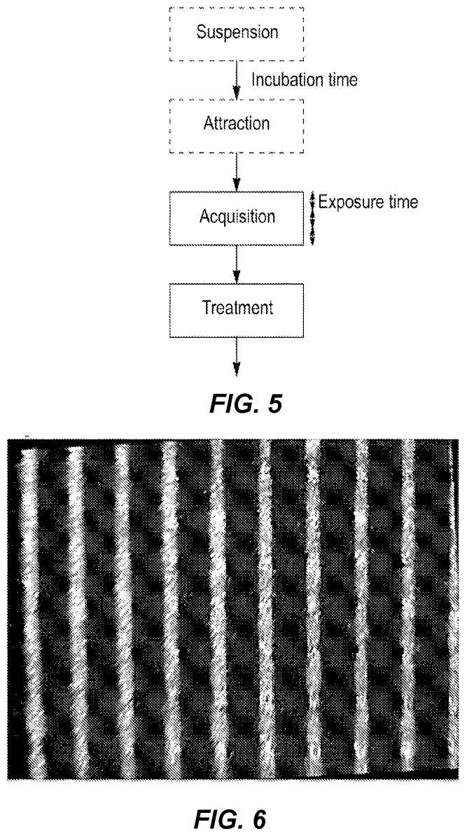

The main steps of the analyzing method are shown in FIG. 5.

Thus, during a first step, the detection elements 10 and the capture elements 9 are respectively suspended in the sample of each chamber 5 to be mixed therein. This suspension may, in particular, comprise a separation of the clusters from the bottom of the chambers 5 as well as a separation of the elements 9, 10 from one another in order to disperse them in the sample. For this purpose, vibration means, for example, a piezoelectric actuator, can be implemented. These vibration means are particularly suitable for imposing a vibration at the bottom of a determined chamber 5 or a plurality of chambers 5 of the cartridge 1. This vibration makes it possible to generate an acoustic pressure field in the liquid present in the analysis chamber, and thus to detach the clusters and suspend the elements 9, 10 forming these clusters. It will be noted that this step must combat the attraction forces present between the magnetic particles of the capture elements 9 and the magnetic layer 6b (screened by the non-magnetic surface film 6c), which is not conventional.

It is pointed out that it is in no way necessary to have provided for placing the capture 9 and detection 10 elements in the form of clusters in the chambers 5 of the cartridge (or in another location of the cartridge) in order to mix them into the sample to be analyzed and, in an alternative embodiment, this mixing is carried out, with the liquid to be analyzed, before introducing this liquid into the cartridge. The preceding step of resuspension is consequently perfectly optional.

Regardless of the way with which these elements 9, 10 are mixed with the liquid, during the following incubation period, and when the analyte is present in the sample, complexes comprising a capture element 9, the analyte, and a detection element 10 are formed.

At the end of the incubation period, the complexes comprising the analyte and a photoluminescent marker are immobilized on the area to be analyzed 6e of the chamber 5 by preferably agglomerating at the magnetic field intensity maxima (that is to say the attraction zones of the area to be analyzed 6e). They are arranged according to the detection pattern defined by the magnetic layer 6b. The excess detection elements 10, i.e., the photoluminescent markers remain suspended in the sample. The non-complexed capture elements 9, which are therefore not associated with detection elements 10, are also immobilized on the area to be analyzed 6e of the chamber 5. In the absence of photoluminescent markers, they cannot however be made visible in the rest of the steps of the analyzing method.

This immobilization can, in particular, be favored during a step of attraction of the magnetic particles comprised in the magnetic complexes and/or in the capture elements 9 present in the sample. During this attraction step, the chamber 5 is exposed to an attraction magnetic field provided by an external magnetic source called an “attraction” source. The attraction magnetic field exacerbates the magnetic field produced by the magnetic layer 6b. It magnetizes the magnetic particles, even those far from the bottom of the chamber, which makes it possible to increase the capture force that applies. It makes it possible to attract and immobilize the complexes on the area to be analyzed 6e, as has been explained in relation to the description of FIG. 4. The field produced by the attraction magnetic source also makes it possible to magnetize the superparamagnetic particles of the sample. In this way, the migration of these particles and complexes is facilitated when they are present proximate the surface of the support 6 in order to immobilize them.

It is possible to perfectly control the duration of the incubation period by activating the attraction magnetic source at the desired moment, so as to place the chamber 5 in the attraction magnetic field.

This attraction magnetic field has, at the area to be analyzed of a chamber, an intensity of between 5 mT and 400 mT, advantageously between 50 mT and 200 mT. A low intensity tends to increase the duration of this step of attraction, and an excessive intensity, for example, greater than 400 mT could exceed the value of the coercive field of the magnetic layer 6b. Also, to preserve the magnetization of this layer and the detection pattern that this defined magnetization, it is preferable to limit the intensity of the attraction magnetic field to below the threshold of 400 mT. When the intensity of the attraction magnetic field is within the preferred range between 50 mT and 200 mT, the attraction step extends for a period of between 20 s and 5 min. The attraction magnetic source is operated, at the end of this period, so that the chamber 5 is no longer exposed to the attraction magnetic field or, at least, not significantly.

The field produced by the attraction magnetic source is preferentially oriented orthogonally to the area to be analyzed 6e in order to add to the field generated by the magnetic layer 6b, and thus to increase the intensity of the magnetic field in the attraction zones Za, and to reinforce the detection pattern, but other directions are possible, in particular, parallel to that surface.

As seen previously, the presence of this field can lead to modifying the in-line arrangement Za of the attraction zones, or, more generally, to redefine the detection pattern, as is encoded by the magnetic layer 6b. The field produced by the attraction magnetic source can be continuous or pulsed, in this case with a pulse duration typically greater than 1 ms, or greater than 10 ms or even 100 ms.

Several approaches are possible for selectively placing a chamber 5 in and outside the magnetic field produced by the attraction magnetic source. The attraction magnetic source can thus be activated electrically. In this case, it may be constituted by an electromagnet, arranged close to the chamber 5. It is then possible to control the attraction magnetic source to “turn on or off” the produced magnetic field as desired. Alternatively, provision may be made for the attraction magnetic source to be able to move relative to the analysis support 6 to be selectively arranged in a first position, in which the chamber is essentially outside the field produced by the attraction magnetic source or be selectively arranged in a second position, in which the chamber is within the field produced by the attraction magnetic source. It is thus possible to choose to move the attraction magnetic source and/or the cartridge.

Just like the re-suspension step (when one is present), the attraction step can be carried out on a single chamber 5 of the cartridge 1, by locating the attraction magnetic field produced by the attraction magnetic source mainly at this chamber 5. Alternatively, provision may be made for the attraction step to be carried out on a plurality of chambers 5 of the cartridge 1 simultaneously, or even on all the chambers 5 of the cartridge 1 simultaneously.

It will be noted that the step of attraction of the magnetic complexes optionally present in the sample to immobilize them at the attraction zones is in no way necessary, a necessary step or limited to what has just been described. It may be provided to immobilize these complexes in attraction zones of an area to be analyzed via other approaches. These complexes can thus be handled by electro-acoustic methods, by means of an acoustic clamp, or by electrophoretic, dibutyl, or even optical methods, to confine them in these zones. These volume forces applied to these particles are respectively induced by the gradients of acoustic, electrical or optical pressure fields, which interact with the particles having different acoustic, dielectric or optical properties from their environment.

It is also possible to arrange the capture elements and/or the detection elements according to a pattern directly on the analysis support, for example, using an inkjet printing or micro-contact printing technique, which makes it possible to properly control the alignment of the magnetic particles of the capture elements. The attraction zones are thus defined very directly. In this alternative approach, the capture elements arranged at the surface of the support react with the analyte (and, optionally, with the detection elements) contained in the biological liquid or microdrops of this liquid discharged or deposited on the surface to form the complexes. This surface reaction may be accelerated by virtue of a magnetic field. The detection elements can be added subsequently to the formation of the complexes, after a possible washing step.

In all cases, and whatever the sequence of steps applied, the presence of an analyte in the sample leads to the formation of magnetic complexes comprising the analyte and a photoluminescent marker on an area to be analyzed of a support and according to a predefined detection pattern.

Continuing the description of the steps composing the analyzing method, the latter comprises a step of acquiring a digital image of the area to be analyzed 6e. In the example taken, the area to be analyzed forms the bottom of a chamber 5 of the cartridge 1. The acquisition of the digital image takes place during an exposure time, using an image capturing device having an optical axis directed toward the area to be analyzed 6e. The area to be analyzed 6e of the chamber 5 is arranged in the depth of field of the image capturing device. During the exposure time, a sensitive surface of the image capturing device is exposed to the light radiation produced by the photoluminescent markers present on the area to be analyzed and in the sample to form a digital image thereof. The photoluminescent markers in solution in the sample or immobilized on the support 6 of the illuminated chamber 5 may be activated by way of the light source and thus made visible in the image plane of the image capturing device. Generally, the characteristics of the light source can be chosen according to the nature of the photoluminescent markers, and, in particular, according to the excitation wavelength of these markers. As an example, the light source may have an excitation wavelength of 650 nm, typically between 600 nm and 700 nm, and the emission wavelength of the markers is on the order of 660 nm.

The exposure time is typically between 5 ms and 1200 ms. The digital image prepared by the image capturing device has a spatial variation in intensity in accordance with the detection pattern when the analyte is present in the sample. The amplitude of this spatial variation is representative of the concentration of the analyst in the sample. An example of such an image is reproduced in FIG. 6.

This acquisition step is followed by a step of processing the digital image to identify therein the spatial variation in intensity, which was briefly presented in the introduction of the present disclosure. This step of processing the digital image seeks, in particular, to measure on this image a specific signal corresponding to the (spatially) average intensity of the light pattern produced by the complexes thus conforming to the attraction zones defined together by the magnetic field produced by the magnetic layer 6b and the field of attraction produced by the external attraction magnetic source. The step of processing the digital image also seeks to measure a non-specific signal (or “supernatant”), corresponding to the (spatially) average intensity of the illuminated background formed of the non-linked detection elements, bearing the photoluminescent markers remaining dispersed in the liquid contained in the chamber 5.

The combination of the specific signal and the supernatant signal makes it possible to determine the presence and/or the concentration of the analyte in the sample of biological fluid, as is, for example, exposed in document EP3447492 presented in the introduction of the present disclosure.

It has been realized that very surprisingly, the intensity of the light radiation produced by the complexes immobilized at the level of the areas of attraction of the area to be analyzed 6e could be significantly improved if the analysis support 6 was arranged in a magnetic field whose properties were perfectly controlled. This observation is all the more surprising since this phenomenon is quite particularly observable when the support is provided with a non-magnetic surface film 6c. According to a non-limiting interpretation of this phenomenon, it seems that the complexes are immobilized, at the end of the attraction step, on the attraction zones in the form of disorganized chains or heaps. This immobilization at the attraction zones in the form of disorganized chains is promoted by the intensity of the gradients generated on the surface of the non-magnetic layer by the underlying magnetic layer. However, these pulling forces are sufficiently moderate to apply an additional magnetic field in order to direct and organize these chains in the same direction so as to make the complexes more visible. The presence of the non-magnetic surface film 6c makes these complexes more sensitive to the presence of the additional magnetic field, due to the separation of the magnetic layer 6b, which tends to reduce the intensity of the gradients. The present disclosure seeks to take advantage of this observation, without however being limited to a cartridge configuration comprising a non-magnetic surface film 6c.

Also, according to an important characteristic, during at least part of the exposure time of the area to be analyzed 6e to the sensitive part of the image capturing device, this area to be analyzed 6e is arranged in a magnetic field called an “illumination” field produced by an illumination magnetic source. This illumination magnetic field is chosen to be parallel to the optical axis of the imaging device over at least part of the area to be analyzed 6e (and preferentially over this area to be analyzed, of course). This field may be oriented toward the image capturing device or in the opposite direction. This part of the area to be analyzed subjected to this illumination field has, on the image produced by the image capturing device, a detection pattern (when the analyte is present in the sample) having an increased intensity and contrast. This intensity can thus be 10 times greater in the presence of the illumination magnetic field parallel to the optical axis of the image capturing device than in the absence of this illumination magnetic field.

For the sake of precision, by “parallel,” it is meant that in the relevant part of the area to be analyzed, the field and the optical axis of the image capturing device are perfectly aligned, to within 15° and preferentially to within 10°, and even more preferentially to within 3°.

The illumination magnetic field has, at the area to be analyzed, any intensity, for example, between 1 mT and 400 mT, advantageously between 10 T and 200 mT, and even more advantageously between 50 mT and 150 mT. Again, it is avoided to apply a field whose intensity could affect the magnetization of the magnetic layer 6b included in the support 6. The illumination magnetic field may be of smaller intensity than that of the attraction magnetic field.

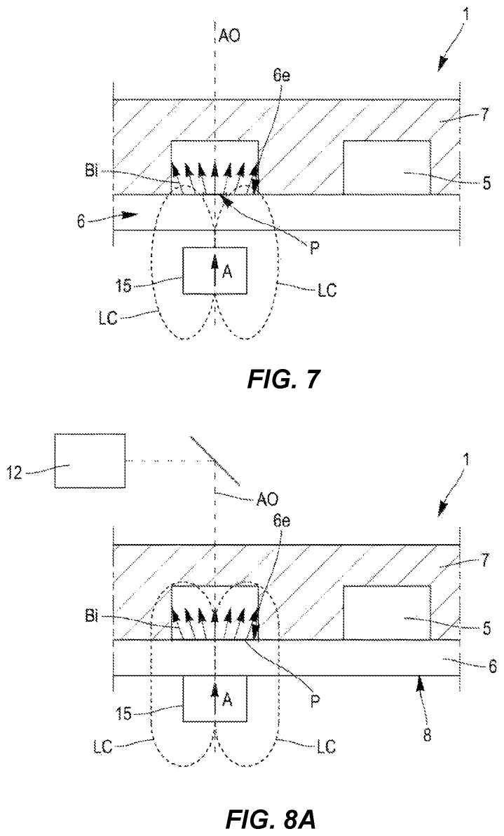

It should be noted that it is neither necessary nor sufficient for the magnetization A of the illumination magnetic source 15 to be directed parallel to the optical axis AO of the imaging device so that it is the case of the magnetic field Bi produced by this source at the area to be analyzed 6e. Indeed, and as is perfectly known and represented by way of illustration in FIG. 7, the field produced by the illumination magnetic source 15 is directed, at any point from the space surrounding the source 15, according to field lines LC, which tend to loop back onto this source 15. Depending on the precise positioning of the illumination magnetic source 15 with respect to the cartridge 1, the illumination magnetic field existing at the area to be analyzed 6e may be quite different, in the direction and in orientation, of those of the magnetization A of the source 15. It is therefore indeed the existing illumination magnetic field Bi at the area to be analyzed 6e (and at least on a part thereof) that it is perfectly necessary to control in order to derive all the benefits of this illumination field. It is thus observed in FIG. 7 that only part P of the area to be analyzed has a field Bi that satisfies the requirements of parallelism with the optical axis.

It will be noted that the cartridge is arranged relative to the image capturing device so that the area to be analyzed 6e of a chamber 5 is generally perpendicular to the optical axis AO of the device (at the location where this optical axis AO intercepts the area to be analyzed 6e). This general arrangement is however limited by the mechanical precision of alignment of the two elements with respect to one another. However, considering that this inaccuracy can become negligible, the alignment characteristic of the magnetic field of illumination with respect to the optical axis of the imaging device can then correspond to this illumination magnetic field being perpendicular to the general plane defined by the area to be analyzed 6e of the cartridge. Again, this condition of perpendicularity is defined to within 15°, preferentially to within 10°, and even more preferentially to within 3°. This assumption of perpendicularity between the area to be analyzed 6e and the optical axis AO of the image capturing device will be retained in the rest of this description, for greater simplicity.

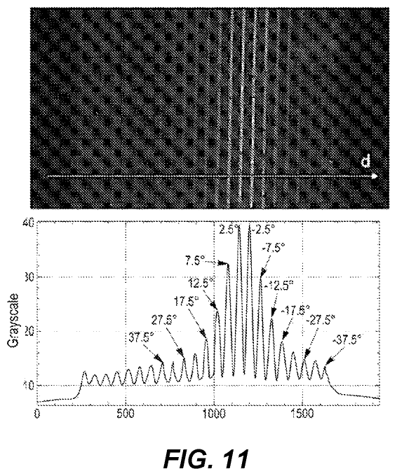

By way of illustration of the benefit provided by the application of an illumination magnetic field having the required property of parallelism with respect to the optical axis, the upper part of FIG. 11 represents an image of an area to be analyzed on which the complexes have been previously immobilized on attraction zones defining a pattern of parallel lines. A magnet was placed under the area to be analyzed during the camera shot that captured this image. The lower part of FIG. 11 shows the light intensity of the image (measured in grayscale) measured along the direction d represented on the image. This intensity changes in “comb” fashion, the vertices of the combs being aligned on the attraction zones in which the complexes are immobilized. The lower part of FIG. 11 also shows, at the intensity vertices, the estimated angle of the magnetic field produced by the magnet with respect to the optical axis, at the area to be analyzed. It is clearly observed that the intensity of the vertices and the contrast of the image are greatly improved when this field is best aligned with the optical axis of the image capturing device. This is particularly visible when this alignment is within 15°.

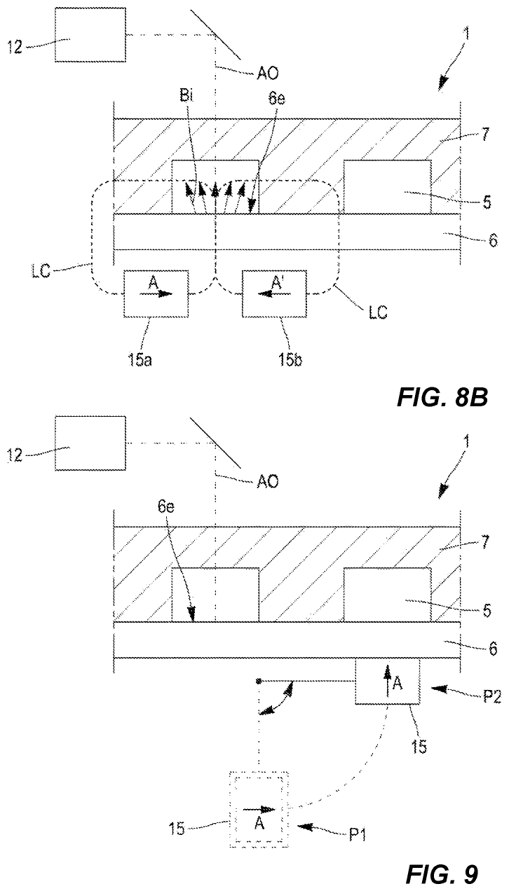

Many configurations are possible to produce this illumination magnetic field during at least part of the exposure time. Thus, according to a first configuration shown in FIG. 8A, the illumination magnetic source 15 is arranged against or near the rear face 8 of the analysis cartridge 1, and precisely under the analysis chamber 5. In this configuration, the magnetization A of the illumination magnetic source 15 can be directed perpendicularly to the area to be analyzed 6e. The illumination magnetic source 15 is positioned against or at a chosen distance from the rear face 8 of the analysis cartridge so that the illumination magnetic field produced by this source is perpendicular to the general plane defined by the area to be analyzed 6e of the cartridge 1 at this surface.

According to another configuration shown in FIG. 8B, the illumination magnetic source 15 is formed of two magnets 15a, 15b arranged under the rear face 8 of the analysis support 6, the magnetization A, A′ of the magnets 15a, 15b being opposite one another and in a direction parallel to the area to be analyzed 6e. The magnets are arranged relative to this cartridge 1 so that the magnetic field Bi produced at the area to be analyzed 6e indeed has the requirement for perpendicularity.

As was already the case of the attraction magnetic source, the illumination magnetic source 15 is operable to selectively place the analysis support 6 within the magnetic field of illumination or outside this magnetic field. For example, this source, the illumination magnetic source 15 or the magnets 15a, 15b forming one of the two configurations presented above, may be electromagnets whose activation and deactivation can be electrically controlled. It is thus possible to selectively control this source 15 to activate it and deactivate it in a coordinated manner with the image capturing device 12 so that, during at least part of the exposure time, the illumination magnetic field is produced. Alternatively, the illumination magnetic source 15 can be mobile relative to the cartridge 1 and to the analysis support 6 of this cartridge 1, to place it selectively in a first position P1 in which the analysis support 6 of the chamber 5 is essentially outside the field produced by the illumination magnetic source 15 or be arranged in a second position P2, in which the analysis support 6 of the chamber 5 is arranged in the field produced by the illumination magnetic source 15.

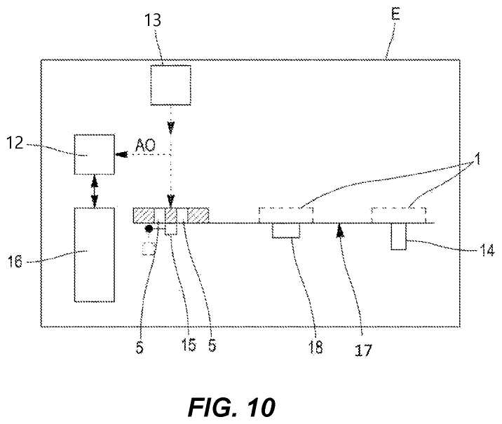

With reference to FIG. 9, the analyzing method comprises in this case the movement of the illumination magnetic source 15 between the first position P1 and the second position P2. Then, at the end of the step of acquiring the digital image, the displacement of the illumination magnetic source 15 from the second position P2 to the first P1. This movement is coordinated with the activation of the image capturing device so that, during at least part of the exposure time, the area to be analyzed 6e is arranged in the illumination magnetic field exhibiting the aforementioned direction. This movement between the first and second positions P1, P2 must be perfectly controlled so as not to invert, during the movement, the orientation of the field Bi at the area to be analyzed 6e. Such a change of orientation could lead to the movement of the complexes immobilized on this surface, and to affect their arrangement outside the detection patterns, which would no longer allow the analysis to be carried out with the desired precision.

Thus, it is preferable that the relative movement of the illumination magnetic source 15 with respect to the area to be analyzed 6e, comprises an approach phase during which the illumination magnetic field Bi, at the area to be analyzed 6e, preserves its direction and its general orientation. The source 15 can thus be moved relative to the support 6 in a direction perpendicular to the area to be analyzed 6e. This approach phase corresponds to the final part of the movement during which these two elements are closest to each other and the area to be analyzed 6e immersed in the magnetic field produced by the illumination magnetic source 15. This avoids the change in direction and orientation of the field.

The movement can thus be entirely conducted, relative to the support, in a direction perpendicular to the area to be analyzed. Alternatively, this movement may comprise any initial phase, this initial phase being carried out while the source 15 and the support 6 are sufficiently distant from each other so that the area to be analyzed 6e is not immersed in the magnetic field produced by the illumination magnetic source 15, or in a very reduced intensity field. It may, for example, involve moving this source 15 along an arc of a circle arranged in a plane perpendicular to the support 6 and under the area to be analyzed of the chamber 5, one end of this arc of a circle forming the approach phase of the illumination magnetic source 15, being perpendicular to this support. This configuration is precisely the one shown in FIG. 9.

It is naturally possible to envisage many other types of movement of the illumination magnetic source 15 and/or of the support 6 making it possible to avoid the change of direction and orientation of the field lines at the area to be analyzed 6e, when the illumination field is applied.

An alternative approach is now presented to that consisting of bringing the source in a direction perpendicular to the area to be analyzed so as to avoid or limit the immobilization of the complexes outside the detection patterns. This alternative approach involves moving this source substantially parallel to the area to be analyzed, so that the orientation of the field Bi at the immobilized complexes performs at least one complete rotation (360°). In this way, the complexes potentially immobilized outside the attraction zones defining the detection patterns are effectively moved toward these attraction zones, by a “sweeping” effect.

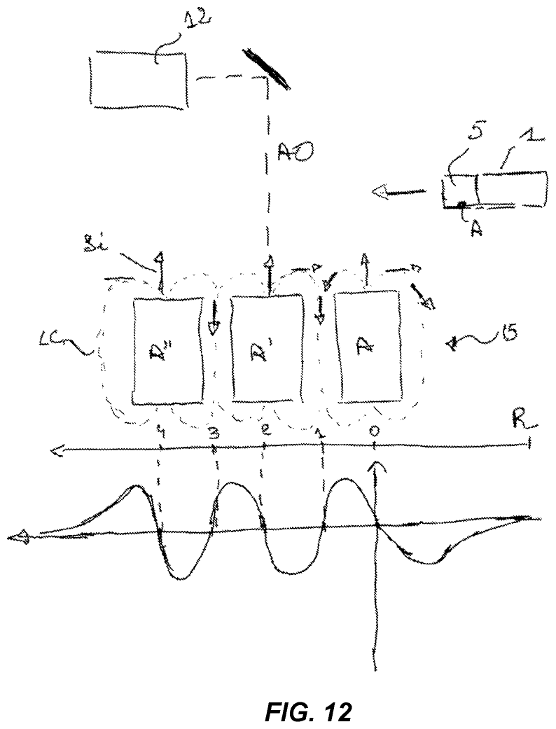

FIG. 12 shows an illumination magnetic source 15 compatible with such an approach. This source 15 is formed of three elementary magnetic sources A, A′, A″ having the same magnetization and separated from each other by a separation distance. By way of example and by repeating the configuration and the dimensions of the cartridge shown in FIGS. 1 and 2, the two elementary sources can be formed by two cylinders having a diameter on the order of 8 mm, a height of 16 mm and separated from each other by a distance of 3 mm. More generally, provision may be made for the illumination magnetic source 15 to be formed from a plurality of elementary sources separated from each other and all having the same orientation.

FIG. 12 shows the lines of the illumination field LC, and vectors representative of this field at different points of the surrounding space. The field is relatively intense between two of the elementary magnetic sources A, A′, A″ and relatively less intense on either side of the external sources A, A″.

When the area to be analyzed of the chamber 5 is moved above the magnetic source, a point of this area to be analyzed is subjected to a rotating field. To illustrate this, a marker R is placed in FIG. 12 linked to the illumination magnetic source 15, this marker R defining an axis of relative movement of the source 15 and the area to be analyzed.

The bottom of FIG. 12 shows the component of the illumination magnetic field Bi along the axis of displacement at a reference point A of the area to be analyzed, when the illumination magnetic source moves to advance the reference point A in the direction of the axis of movement. This component is capable of generating a movement of the immobilized complexes, interacting with their magnetic part.

It is desired to place the reference point A at the marker 2, the illumination field Bi generated by the elementary sources having the qualities required to carry out the digital acquisition step in this position. The forces that apply to the complexes during this movement tend to accumulate these complexes on at least one area of attraction of the area to be analyzed. This is, in particular, the case at the end of the relative displacement of the reference point A from its starting point shown in FIG. 12 to the marker 2.

It should be noted that it is also possible to align the optical axis AO on the marker 3 and to move the reference point A at this marker 3. Indeed, the illumination field Bi generated by the elementary sources also has at this marker the qualities required to carry out the digital acquisition step.

It is therefore possible by suitably configuring the source 15 to relatively move the source and the area to be analyzed, in a direction parallel to this surface.

In all the embodiments that have just been described, the movement step is coordinated to the step of acquisition of the digital image, so that during at least part of the acquisition period, the area to be analyzed 6e (or part thereof) of the chamber 5 is immersed in the illumination field Bi having the required direction and orientation characteristics.

It will be noted that the positioning of the illumination magnetic source 15 with respect to the support 6 making it possible to produce an illumination field Bi having these required characteristics is particularly sensitive. Also, in some cases, it may be advantageous to provide during the acquisition step a positioning sub-step during which the relative position of the illumination magnetic source 15 with respect to the analysis support 6 is adjusted. During this positioning sub-step, successive digital images are acquired, the intensity of which can be measured in order to determine the optimum relative positioning. In other words, the step of acquiring the digital image comprises a plurality of exposure periods to establish, respectively, a plurality of digital images. These digital images can be used to determine the best relative position between the illumination magnetic source 15 and the support 6, i.e., that having a detection pattern of better quality.

When it is not possible or it is difficult to control the illumination magnetic field Bi so that the field has the required direction characteristic over the entire extent of the area to be analyzed 6e of a chamber 5, and therefore so that these conditions are obtained only for a part of this area to be analyzed 6e, it is also possible to take advantage of the positioning sub-step and the multiple digital images acquired during the acquisition step to combine them together and ultimately obtain a detection pattern of good quality over the entire area to be analyzed 6e of the chamber 5, or a large part of this area to be analyzed 6e.

According to a very advantageous approach, and when the method provides an attraction step implementing an attraction magnetic source as described above, the same magnetic source can be used both for the attraction step and during the step of acquisition of a digital image to provide the illumination magnetic field. In such a case, this single magnetic source must be such that the magnetic field produced has the required characteristic of the illumination field Bi, that is to say parallel to the optical axis AO of the image capturing device 12. It is thus possible to provide, in addition to their direction and their orientation, these two fields are precisely identical, in particular, in intensity. This approach is very advantageous in that it avoids moving the cartridge 1 forming a support to position it successively in two different fields. The single field of attraction and illumination can be activated and maintained at the end of the incubation step to, initially, immobilize the complexes on the area to be analyzed 6e, then allow the progress of the acquisition step thus to form at least one high-quality digital image.

Alternatively to this advantageous approach, the attraction magnetic source and the illumination magnetic source 15 may be different. In this configuration, advantageously, the attraction magnetic field and the illumination magnetic field have the same direction or a very similar direction as well as the same orientation at the part of the area to be analyzed 6e. This avoids rearranging the complex strings and/or moving them by going from one field to another. A transfer step can be provided during which the cartridge 1 is moved, between the attraction step and the acquisition step, from an incubation position where it can be subjected to the field produced by the attraction magnetic source to an acquisition position where it can be subjected to the illumination field produced by the illumination magnetic source 15.

In order to fully control the phenomena that occur in an analysis chamber during the incubation period and detect the presence and intensity of the detection pattern after this period, it is advantageous to place the cartridge 1 in or on an analysis device E, one embodiment of which is shown schematically in FIG. 10.

Analysis Device

The analysis device E aims to implement the method that has just been explained. All the features disclosed in the presentation of this method can therefore be incorporated into this device. For the sake of conciseness, only the main features of this device are therefore described here.

The device E comprises a host support for receiving the cartridge 1 in order to position it as precisely as possible in an acquisition position. In this position, at least one chamber 5 of the cartridge 1 is arranged in the field of an image capturing device 12, such as an image sensor. This chamber 5 is also arranged in the illumination light field of a light source 13, for example, a light-emitting diode-based source. It is also possible to provide for the optical path between the light source 13, the chamber 5 and the image capturing device 12 of the optical elements such as separators, filters, lenses in order to improve the quality of the picture taking and, in particular, to choose suitable magnification and depth of field. It is possible, with this arrangement, to acquire a digital image of the sample and of the support 6 of the chamber 5, in order to reveal on the image the light intensity produced by the fluorescent markers. The cartridge 1 is of course arranged in the analysis device so that the upper cover 7, transparent at least in front of the chambers 5, is in the optical path in order to allow this image capture. Advantageously, the host support of the cartridge is configured so that the area to be analyzed 6e forming the bottom of the chambers of the cartridge 1, here formed of the non-magnetic surface film 6c, is perpendicular to the optical axis AO of the image capturing device 12. Provision may be made for the host support to be able to move so as to position a single chamber 5 or a plurality of chambers 5 of the cartridge 1 very precisely in the acquisition position. It is possible to treat it during successive operations, all chambers 5 of the cartridge.

The analysis device E of FIG. 10 also comprises an illumination magnetic source 15 able to produce the illumination magnetic field to which the analysis support is exposed when the support is in the acquisition position. As already stated, the illumination magnetic field is parallel to the optical axis AO (at the location where this axis intercepts the area to be analyzed 6e) and oriented toward the image capturing device 12 over at least part of the area to be analyzed 6e.

In the advantageous configuration of FIG. 10, the image capturing device 12 is arranged on one side of the host support, and the illumination magnetic source 15 is arranged on the other side of the host support. This arrangement makes it possible to place the cartridge 1 between the illumination magnetic source 15 and the image capturing device 12. In particular, it makes it possible to position the illumination magnetic source 15, close or even in contact with the rear face 8 of the cartridge 1. All the benefits of such a configuration were seen above.

The illumination magnetic source 15 may be movable to selectively place the host support (and therefore the cartridge when one is present) in the illumination magnetic field or place the host support outside the illumination magnetic field. The source 15 can also be controllable to selectively produce the illumination magnetic field and interrupt it. It may, in particular, be an electromagnet. It is also possible to imagine that the illumination magnetic source 15 is both mobile and controllable.

In operation, the photoluminescent markers in solution in the sample or immobilized on the area to be analyzed 6e of the chamber 5 are activated by means of the light source 13 and made visible in the image plane of the image capturing device 12. A digital image of the distribution can thus be acquired in the plane of the support of the photoluminescent markers.

As has already been mentioned, the illumination magnetic source 15 and the field generated by this source can also be used to attract and immobilize the complexes on the area to be analyzed of a chamber 5 of the cartridge, during an attraction step.

Continuing the description of the analysis device E of FIG. 5, this may also optionally comprise vibration means 14, for example, a piezoelectric actuator, capable of coming into contact with the support 6 of the cartridge 1, particularly under a determined chamber 5, in order to apply vibratory forces thereto. The actuator 14 can be activated after the cartridge 1 is inserted into or onto the device E so as to allow effective resuspension of the capture elements, magnetic particles and elements 9, 10 in the sample, as was previously described.

The device E may comprise an attraction magnetic source 18, for example, a magnet or an electromagnet, distinct from the illumination magnetic source. This source can be activated in such a way as to exacerbate the magnetic field produced by the magnetic layer 6b and to make it possible to attract and immobilize the complexes on the area to be analyzed 6e.

In this dual-source configuration that is shown in FIG. 10, provision may be made for the host support to be movable to allow the cartridge 1 to be moved from a position in which it can be exposed to the field of attraction produced by the source of attraction, to another position where it can be exposed to the field produced by the illumination magnetic source 15 and placed in the depth of field of the image capturing device 12. This support may, for example, be controlled to slide along a transfer rail 17 along which certain elements composing the device E are arranged. It is thus possible to provide for the analysis support 6 to be movable along the transfer rail 17 from an incubation position where it can be subjected to the field produced by the attraction magnetic source 18 to the acquisition position where it can be subjected to the illumination magnetic field produced by the illumination magnetic source 15. Provision may also be made for the piezoelectric actuator 14 to be arranged along the transfer rail to enter into action while the cartridge 1 lies in a position distinct from the acquisition position and from the incubation position, as is the case in FIG. 10.

In order to operate the analysis device E according to the method previously presented, and possibly to carry out the digital processing of the image that has been acquired, the device E also comprises a computing device 16. This may be a microcontroller, a microprocessor, an FPGA circuit. In addition to the computing means strictly speaking, the computing device 16 also comprises memory components making it possible to store data and computer programs enabling the device E to be operated. The computing device 16 can also comprise interface components for exchanging data (of the USB interface type or of the short- or long-range wireless type such as Wi-Fi, Bluetooth, 3G, LORA, Sigfox, etc.) or making it possible to connect the analysis device E to maintenance equipment. The interface components can also comprise a screen and control buttons to allow the use of the device E by an operator. The computing device 16 is connected, for example, by means of an internal bus, to the image capturing device 12, to the light source 13, to the mechanical actuator 14, to the illumination magnetic source 15, and potentially the attraction magnetic source, in order to coordinate their actions and/or to collect the data produced, for example, the digital images provided by the image capturing device 12.

When the cartridge 1 contains a plurality of chambers 5, the operations implemented by the computing device 16 can be carried out successively on the analysis chambers 5 of the cartridge 1. Alternatively, these operations can be carried out simultaneously on a plurality or all of the chambers 5 of the cartridge 1.

As will be readily understood, the present disclosure is not limited to the described embodiment, and it is possible to add variants thereto without departing from the scope of the invention as defined by the claims.

1. A method for analyzing a liquid that may contain an analyte, a sample of the liquid being arranged on an area to be analyzed of an analysis support, the area to be analyzed having a plurality of attraction zones arranged according to a detection pattern, the sample including magnetic complexes comprising the analyte and a photoluminescent marker immobilized at the attraction zones, and/or supernatant photoluminescent markers, the analyzing method comprising:
acquiring a digital image of the area to be analyzed during an exposure time, using an image capturing device having an optical axis directed toward the area to be analyzed, the digital image having a spatial variation in intensity in accordance with the detection pattern when the analyte is present in the sample; and
processing the digital image to identify therein the spatial variation in intensity;
wherein, during at least part of the exposure time, the analysis support is arranged in a magnetic illumination field produced by an illumination magnetic source, the illumination magnetic field being parallel to the optical axis over at least part of the area to be analyzed.

2. The method of claim 1, wherein the illumination magnetic source is operable to place the analysis support in the magnetic illumination field, the method further comprising moving the analysis support relative to the illumination magnetic source to selectively place the analysis support within and outside the magnetic illumination field.

3. The method of claim 2, wherein the moving of the analysis support relative to the illumination magnetic source is controlled so as not to change a direction or reverse an orientation of the magnetic illumination field at the area to be analyzed.

4. The method of claim 2, wherein the moving of the analysis support relative to the illumination magnetic source is controlled so that the orientation of the illumination field undergoes at least one complete rotation.

5. The method of claim 1, wherein the acquiring of the digital image comprises a plurality of exposure periods to establish, respectively, a plurality of digital images, and the method comprises, between two exposure periods of the plurality of exposure periods, modifying the relative position of the illumination magnetic source with respect to the analysis support.

6. The method of claim 1, wherein the sample includes the sample including magnetic complexes, and the method further comprises, before the acquiring of the digital image, attracting the magnetic complexes to the attraction zones and immobilizing the magnetic complexes at the attraction zones.

7. The method of claim 6, wherein the attracting the magnetic complexes to the attraction zones comprises exposing the analysis support to an attraction magnetic field provided by the illumination magnetic source.

8. The method of claim 6, wherein the attracting the magnetic complexes to the attraction zones comprises exposing the analysis support to an attraction magnetic field produced by an attraction magnetic source distinct from the illumination magnetic source.

9. The method of claim 8, wherein the attraction magnetic field and the magnetic illumination field have the same directions and the same orientation at the area to be analyzed.

10. The method of claim 1, wherein the analysis support comprises a magnetic layer defining at least part of the attraction zones and a non-magnetic surface film arranged on the magnetic layer, the magnetic surface film defining the area to be analyzed.

11. An analysis device comprising:
a host support for receiving and placing, in an acquisition position, an area to be analyzed of an analysis support and a plurality of attraction zones arranged according to a detection pattern on the area to be analyzed;
an image capturing device having an optical axis and a depth of field, the image capturing device being arranged to receive the area to be analyzed in a depth of field of the image capturing device when the analysis support is in the acquisition position; and
an illumination magnetic source configured to produce an illumination magnetic field to which the analysis support is exposed when that support is in the acquisition position, the illumination magnetic field being parallel to the optical axis over at least part of the area to be analyzed.

12. The analysis device of claim 11, wherein the image capturing device is arranged on one side of the host support, and the illumination magnetic source is arranged on the other side of the host support.

13. The analysis device of claim 12, wherein the illumination magnetic source is movable to selectively place the host support in the illumination magnetic field or place the host support outside the illumination magnetic field.

14. The analysis device of claim 13, further comprising an attraction magnetic source distinct from the illumination magnetic source, the attraction magnetic source configured to produce an attraction magnetic field.

15. The analysis device of claim 14, further comprising a transfer rail for moving an analysis support from an incubation position where the analysis support can be subjected to the attraction magnetic field produced by the attraction magnetic source to the acquisition position where the analysis support can be subjected to the illumination magnetic field produced by the illumination magnetic source.

16. The analysis device of claim 15, further comprising an analysis support arranged on the host support, the analysis support comprising a magnetic layers at least partly defining the attraction zones and a non-magnetic surface film arranged on the magnetic layer, the magnetic surface film defining the area to be analyzed.

17. The analysis device of claim 11, wherein the illumination magnetic source is movable to selectively place the host support in the illumination magnetic field or place the host support outside the illumination magnetic field.

18. The analysis device of claim 11, further comprising an attraction magnetic source distinct from the illumination magnetic source, the attraction magnetic source configured to produce an attraction magnetic field.

19. The analysis device of claim 18, further comprising a transfer rail for moving an analysis support from an incubation position where the analysis support can be subjected to the attraction magnetic field produced by the attraction magnetic source to the acquisition position where the analysis support can be subjected to the illumination magnetic field produced by the illumination magnetic source.

20. The analysis device of claim 11, further comprising an analysis support arranged on the host support, the analysis support comprising a magnetic layer at least partly defining the attraction zones and a non-magnetic surface film arranged on the magnetic layer, the magnetic surface film defining the area to be analyzed.
